(19) United States
(12) Patent Application Publication
(10) Pub. No.: US 2011/0318413 A1
(43) Pub. Date: Dec. 29, 2011

(54) EXTENDED RELEASE FORMULATIONS CONTAINING DARIFENACIN OR PHARMACEUTICALLY ACCEPTABLE SALTS THEREOF

(75) Inventors: Rajesh Kshirsagar, Bangalore (IN); Ganesh Shinde, Bangalore (IN); Pravin Kamble, Bangalore (IN)

(73) Assignee: MICRO LABS LIMITED, Bangalore (IN)

(21) Appl. No.: 13/166,446

(22) Filed: Jun. 22, 2011

(30) Foreign Application Priority Data
Jun. 23, 2010 (IN) 1766/CHE/2010

(51) Int. Cl. A61K 31/4025; A61K 9/28; A61K 9/36; A61K 9/00

(52) U.S. Cl. 424/465; 514/422; 424/400; 424/484; 424/488; 424/487

(57) ABSTRACT

The present invention relates to an extended release formulation comprising darifenacin or pharmaceutically acceptable salts thereof and one or more release controlling hydrophobic materials optionally coated with combination of one or more release controlling hydrophobic materials and one or more release controlling hydrophilic materials; wherein said formulation provides controlled release of the darifenacin over the period of 24 hours. The present invention further relates to an extended release formulation comprising darifenacin or pharmaceutically acceptable salts thereof, wherein darifenacin is incorporated in a matrix comprising one or more release controlling hydrophobic materials and one or more release controlling hydrophilic materials.

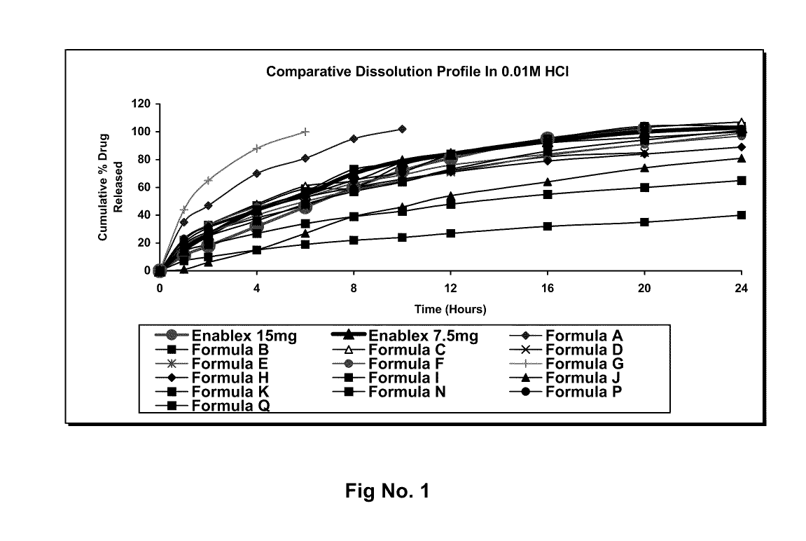

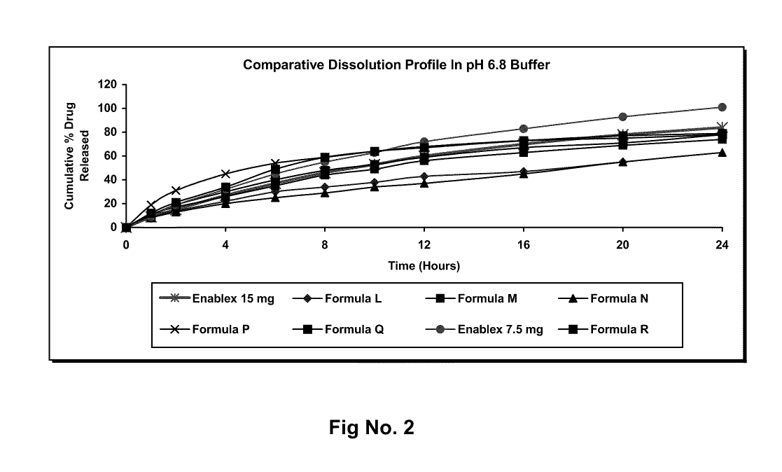

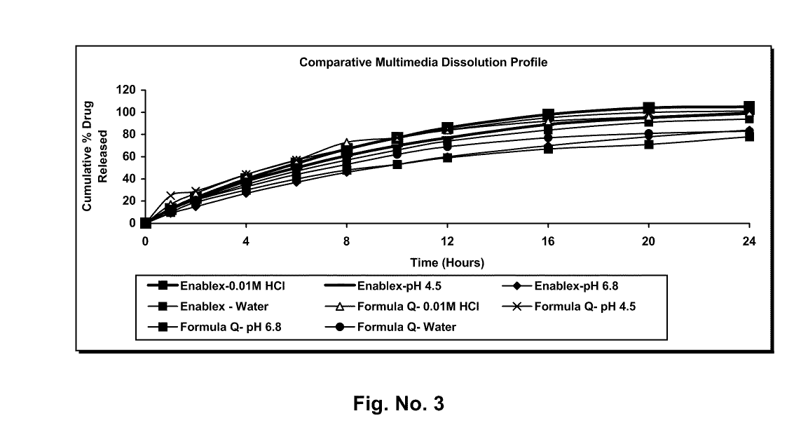

### TECHNICAL FIELD OF THE INVENTION

The present invention relates to an extended release formulation comprising darifenacin, or pharmaceutically acceptable salts thereof and a process for preparing the same.

### BACKGROUND OF THE INVENTION

Darifenacin is (S)-2-{1-[2-(2,3-dihydrobenzofuran-5-yl)ethyl]-3-pyrrolidinyl}-2,2-diphenylacetamide and is disclosed in European Patent No. 0388054, Examples 1B and 8, and is referred to therein as 3-(S)-(−)-(1-carbamoyl-1,1-diphenylmethyl)-1-[2-(2,3-dihydrobenzofuran-5-yl)ethyl]pyrrolidine. It is indicated in the treatment of urinary incontinence and irritable bowel syndrome.

Very few methods are already known for the industrial preparation of extended release oral dosage forms comprising darifenacin, or the pharmaceutically acceptable salts thereof with desirable dissolution profile and a cost effective manufacturing process.

U.S. Pat. No. 6,106,864 discloses a pharmaceutical dosage form adapted for administration to the gastrointestinal tract of a patient, comprising darifenacin, or a pharmaceutically acceptable salt thereof, and a pharmaceutically acceptable adjuvant, diluent or carrier, characterized in that the dosage form is adapted to deliver at least 10% by weight of the darifenacin, or the pharmaceutically acceptable salt thereof, to the lower gastrointestinal tract of the patient. However this patent only discloses formulations with hydroxypropylmethyl cellulose as release controlling agent.

United States patent application No. 2009/0022807 A1 discloses a composition containing beads, with an inert core; a seal layer containing a non-polymeric hydrophobic materials; active ingredient containing layer and release-controlling polymer.

Thus there is need in the art for an extended release formulations comprising darifenacin, or pharmaceutically acceptable salts thereof which are easy to manufacture, comparatively cheaper, bioequivalent and having higher manufacturing yield compared to marketed formulation.

### SUMMARY OF THE INVENTION

It is, therefore, an object of the present invention to provide an extended release formulation comprising darifenacin, or pharmaceutically acceptable salts thereof, one or more release controlling hydrophobic materials and one or more pharmaceutically acceptable excipients wherein the said formulation provides controlled release of the darifenacin over the period of 24 hours.

In yet another aspect the present invention provides an extended release formulation comprising darifenacin or pharmaceutically acceptable salts thereof, wherein darifenacin is incorporated in a matrix comprising one or more release controlling hydrophobic materials optionally coated with combination of one or more release controlling hydrophobic materials and one or more release controlling hydrophilic materials.

In yet another aspect the present invention provides an extended release formulation comprising darifenacin or pharmaceutically acceptable salts thereof, wherein darifenacin is incorporated in a matrix comprising one or more release controlling hydrophobic materials and one or more release controlling hydrophilic materials.

In yet another aspect the present invention provides an extended release formulation comprising darifenacin or pharmaceutically acceptable salts thereof, one or more release controlling hydrophobic materials and one or more pharmaceutically acceptable excipients, which is bioavailable and effective with sufficient shelf-life, good pharmaceutical properties, enhancing patient compliance and reducing possible side effects.

In yet another aspect the present invention provides an extended release solid dosage form for oral administration comprising darifenacin or pharmaceutically acceptable salts thereof, one or more release controlling hydrophobic materials and one or more pharmaceutically acceptable excipients, which can be prepared in dosage forms of different strength by proportionally adjusting the quantities of the excipients and the active ingredient, thereby providing a pharmaceutical linearity, without affecting the dissolution profile and bioavailability of the active ingredient.

In yet another aspect the present invention provides a method for the preparation of an extended release formulation comprising darifenacin or pharmaceutically acceptable salts thereof, one or more release controlling hydrophobic materials and one or more pharmaceutically acceptable excipients thereby enhancing the release rate of the active ingredient and being stable over a long period of time and improving the pharmaceutical characteristics of the formulation.

### BRIEF DESCRIPTION OF THE DRAWINGS

FIG. No. 1: shows comparative dissolution profiles of the pharmaceutical formulations according to the present invention in 0.01M HCl.

FIG. No. 2: shows comparative dissolution profiles of the pharmaceutical formulations according to the present invention in pH 6.8 phosphate buffer.

FIG. No. 3: shows comparative Multimedia dissolution profiles of formulation Q in comparison with Enablex.

### DETAILED DESCRIPTION OF THE INVENTION

The present invention is directed towards, an extended release formulation comprising darifenacin or pharmaceutically acceptable salts thereof, one or more release controlling hydrophobic materials and one or more pharmaceutically acceptable excipients, which can be prepared in dosage forms of different strength by proportionally adjusting the quantities of the excipients and the active ingredient, thereby providing a pharmaceutical linearity, without affecting the dissolution profile and bioavailability of the active ingredient.

The formulation of the present invention provides controlled release of darifenacin along with controlled release action for extended period which is further easy to manufacture, simple and gives a higher yield with a dissolution pattern similar to the marketed formulation.

The term “Darifenacin” herein refers to Darifenacin in the form of freebase or its pharmaceutically acceptable salt(s), hydrate(s), solvate(s) and physiologically functional derivative(s) and precursors thereof. The term also includes all polymorphic forms, whether crystalline or amorphous.

The term “formulation” herein refers to the combination of one or more drug substances and one or more excipients, “Drug product”, “pharmaceutical dosage form”, “dosage form,” “final dosage form” and the like, refer to a pharmaceutical formulation that is administered to a subject in need of treatment and generally may be in the form of tablets, capsules, tablets filled in capsule, mini tablets filled in capsule, sachets containing powder or granules, pellets, liquid solutions or suspensions, patches and the like.

The term “extended release formulation” herein refers to any formulation or dosage form that comprises an active drug and which is formulated to provide a longer duration of pharmacological response after administration of the dosage form than is ordinarily experienced after administration of a corresponding immediate release formulation comprising the same drug in the same amount. Controlled release formulations include, inter alia, those formulations described elsewhere as “controlled release”, “delayed release”, “sustained release”, “prolonged release”, “programmed release”, “time release” and/or “rate controlled” formulations or dosage forms.

By “pharmaceutically acceptable” herein refers to a carrier comprised of a material that is not biologically or otherwise undesirable.

The hydrophobic materials according to present invention includes but are not limited to ethyl cellulose, cellulose acetate, cellulose acetate phthalate, polymethacrylates, calcium silicates, hydrogenated castor oil, hydrogenated vegetable oil, bees wax, carnauba wax, glyceryl distearate, glyceryl palmitostearate, Kollidon SR, and combination thereof and the like. The hydrophobic material preferably present from about 1% w/w to about 98% w/w of formulation, still preferably from about 10% w/w to about 98% w/w of formulation. The most preferred is hydrogenated castor oil or hydrogenated vegetable oil.

The hydrophilic materials according to present invention includes but are not limited to hydroxypropylmethyl cellulose, hydroxypropyl cellulose, methylcellulose, povidone, polyethylene glycols, vinyl acetate copolymers, polysaccharides such as alginates, xanthan gum, chitosan, carrageenan, dextran, polyalkylene oxides such as polyethylene oxide, methacrylic acid copolymers, maleic anhydride/methyl vinyl ether copolymers, carbomers and combinations thereof.

The formulation according to present invention will, in general comprise of one or more excipients. Examples of pharmaceutically acceptable excipients include, but are not limited to binders, fillers or diluents, lubricants, glidants, disintegrants. A combination of excipients may also be used. The amount of excipient(s) employed will depend upon how much active agent is to be used. One excipient can perform more than one function.

Binders include, but are not limited to, starches such as potato starch, wheat starch, corn starch; microcrystalline cellulose such as products known under the registered trade marks Avicel, Filtrak, Heweten or Pharmacel; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations thereof and other materials known to one of ordinary skill in the art and mixtures thereof.

Fillers or diluents, which include, but are not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants may be selected from, but are not limited to, those conventionally known in the art such as Mg, Al, Ca, Zn stearate, polyethylene glycol, glyceryl behenate, mineral oil, sodium stearyl fumarate, stearic acid, hydrogenated vegetable oil and talc.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

The formulation according to present invention may also comprise a disintegrant which may be included in all or part of the oral dosage form to ensure rapid disintegration of the dosage form or part of the dosage form (for example, one of the layers in a bilayer tablet) after administration.

Disintegrants include, but are not limited to: alginic acid, carboxymethylcellulose calcium, carboxymethylcellulose sodium, croscarmellose sodium, crospovidone, guar gum, magnesium aluminium silicate, sodium alginate, sodium starch glycolate and starches and other materials known to one of ordinary skill in the art and combinations thereof.

The formulations of the present invention can optionally have one or more coatings such as film coating, sugar coating, extended release coating, enteric coating, bioadhesive coating and other coatings known in the art. These coatings help formulations to release the drug at and for the required time. The most preferable coating is extended release coating. The coating is present from about 1% w/w to about 50% w/w of the total composition weight, preferably from about 1% w/w to about 15% w/w. In addition to coating ingredients, sometimes commercially available pre-mixed coating materials such as Opadry® Clear 03K19229 (contains hydroxypropylmethyl cellulose, triacetin and talc), Opadry® Clear YS-1R-7006 (contains hydroxypropylmethyl cellulose, PEG 400 and PEG 6000), Opadry® White OY 58900 (contains hydroxypropylmethyl cellulose, PEG 400, and titanium dioxide), and Lusterclear®, etc. will be used. These typically require only mixing with a liquid before use. The preferred coating composition contains the combination of ethylcellulose and Opadry® Clear YS-1R-7006.

These coating comprises one or more excipients selected from the group comprising plasticizers, coating agents, opacifiers, fillers, polishing agents, colouring agents, anti-tacking agents and the like.

The extended release formulation according to present invention may be formulated in order to obtain dissolution independent of pH. The preferred manner to achieve such dissolution is to add a pharmaceutically acceptable organic acid into the dosage form according to the methods known to one skilled in the art.

These pharmaceutically acceptable organic acids can be chosen from example among maleic acid, tartaric, malic, fumaric, lactic, citric, adipic or succinic acid and their acid salts where these exist in the form of recemates or isomers. According to the invention, acid particularly preferred are citric, tartaric, fumaric and succinic and their acid salts.

The various embodiments of the present invention can be assembled in several different ways.

In one embodiment, the present invention provides an extended release formulation comprising;

darifenacin or pharmaceutically acceptable salts thereof;
one or more release controlling hydrophobic materials;
and one or more pharmaceutically acceptable excipients.

In yet another embodiment the present invention provides an extended release formulation comprising;

darifenacin, or pharmaceutically acceptable salts thereof;
one or more release controlling hydrophobic materials;
and one or more pharmaceutically acceptable excipients;
wherein the said formulation provides controlled release of the darifenacin over the period of 24 hours.

In yet another embodiment the present invention provides an extended release formulation comprising darifenacin, or pharmaceutically acceptable salts thereof, one or more release controlling hydrophobic materials and one or more pharmaceutically acceptable excipients; optionally coated with combination of one or more release controlling hydrophobic materials and one or more release controlling hydrophilic materials; wherein an in-vitro dissolution rate when measured using the USP Type I (Basket Apparatus) at 100 rpm in 900 ml, 0.01M hydrochloric acid at 37° C.;

less than 40% darifenacin released after 1 hour;
from about 25% to about 65% darifenacin released after 4 hours;
from about 50% to about 85% darifenacin released after 8 hours;
from about 55% to about 100% darifenacin released after 12 hours;
from about 70% to about 100% darifenacin released after 16 hours;
and greater than 85% darifenacin released after 24 hours.

In yet another embodiment the present invention provides an extended release formulation comprising darifenacin or pharmaceutically acceptable salts thereof, one or more release controlling hydrophobic materials and one or more pharmaceutically acceptable excipients wherein formulation comprises from about 1.5% w/w to about 50% w/w of darifenacin or pharmaceutically acceptable salts thereof.

In yet another embodiment the present invention provides an extended release formulation comprising darifenacin or pharmaceutically acceptable salts thereof, one or more release controlling hydrophobic materials and one or more pharmaceutically acceptable excipients wherein hydrophobic material is present from about 1% w/w to about 98% w/w of the formulation; preferably from about 10% w/w to about 98% w/w of the formulation.

In yet another embodiment the present invention provides an extended release formulation comprising darifenacin or pharmaceutically acceptable salts thereof, incorporated in a hydrophobic matrix comprising one or more hydrophobic materials wherein hydrophobic materials can be selected from group consisting of ethyl cellulose, cellulose acetate, cellulose acetate phthalate, polymethacrylates, calcium silicates, hydrogenated castor oil, hydrogenated vegetable oil, bees wax, carnauba wax and combination thereof.

In yet another embodiment the present invention provides an extended release formulation comprising darifenacin or pharmaceutically acceptable salts thereof, wherein darifenacin is incorporated in a matrix comprising one or more release controlling hydrophobic materials and one or more release controlling hydrophilic materials.

In yet another embodiment the present invention provides an extended release formulation comprising darifenacin or pharmaceutically acceptable salts thereof, wherein darifenacin is incorporated in a matrix comprising one or more release controlling hydrophobic materials optionally coated with combination of one or more release controlling hydrophobic materials and one or more release controlling hydrophilic materials.

In yet another embodiment the present invention provides an extended release formulation comprising darifenacin or pharmaceutically acceptable salts thereof, incorporated in a matrix comprising one or more release controlling hydrophobic materials and one or more pharmaceutically acceptable excipients wherein formulation comprises from about 1.5% w/w to about 50% w/w of darifenacin or pharmaceutically acceptable salts thereof.

In yet another embodiment the present invention provides an extended release formulation comprising darifenacin or pharmaceutically acceptable salts thereof, incorporated in a matrix comprising one or more release controlling hydrophobic materials and one or more pharmaceutically acceptable excipients wherein hydrophobic material is present from about 1% w/w to about 98% w/w of the formulation; preferably from about 10% w/w to about 98% w/w of the formulation.

In yet another embodiment the present invention provides an extended release formulation comprising darifenacin or pharmaceutically acceptable salts thereof, incorporated in a matrix comprising hydrogenated vegetable oil or hydrogenated castor oil wherein formulation optionally coated with combination of ethylcellulose and mixture of hydroxypropylmethyl cellulose and polyethylene glycol.

In yet another embodiment the present invention provides an extended release formulation comprising darifenacin or pharmaceutically acceptable salts thereof, incorporated in a matrix comprising combination of hydrogenated vegetable oil or hydrogenated castor oil and ethylcellulose wherein formulation optionally coated with combination of ethylcellulose and mixture of hydroxypropylmethyl cellulose and polyethylene glycol.

In yet another embodiment the present invention provides an extended release formulation comprising darifenacin or pharmaceutically acceptable salts thereof, incorporated in a matrix comprising ethylcellulose and polyethylene glycol wherein formulation optionally coated with combination of ethylcellulose and mixture of hydroxypropylmethyl cellulose and polyethylene glycol.

In yet another embodiment the present invention provides an extended release matrix tablet comprising darifenacin or pharmaceutically acceptable salts thereof, incorporated in a hydrophobic matrix comprising one or more release controlling hydrophobic materials and one or more pharmaceutically acceptable excipients wherein formulation comprises from about 1.5% w/w to about 50% w/w of darifenacin or pharmaceutically acceptable salts thereof and hydrophobic material from about 1% w/w to about 98% w/w of the formulation; preferably from about 10% w/w to about 98% w/w of the formulation.

In yet another embodiment the present invention provides an extended release tablet comprising darifenacin or pharmaceutically acceptable salts thereof, incorporated in a matrix comprising hydrogenated vegetable oil and one or more pharmaceutically acceptable excipients; wherein matrix is optionally coated with combination of ethylcellulose and mixture of hydroxypropylmethyl cellulose and polyethylene glycol.

In yet another embodiment the present invention provides an extended release matrix tablet comprising;
a) from about 1.5% w/w to about 50% w/w of formulation darifenacin or pharmaceutically acceptable salts thereof;
b) from about 1% w/w to about 98% w/w of formulation of hydrogenated vegetable oil or hydrogenated castor oil;
c) one or more pharmaceutically acceptable excipients;
d) optionally coated with combination of ethylcellulose and mixture of hydroxypropylmethyl cellulose and polyethylene glycol.

In yet another embodiment the present invention provides an extended release matrix tablet comprising;
a) 1.5% w/w to about 50% w/w of formulation darifenacin or pharmaceutically acceptable salts thereof;
b) from about 10% w/w to about 98% w/w of formulation of hydrogenated vegetable oil or hydrogenated castor oil;
c) one or more pharmaceutically acceptable excipients;
d) optionally coated with combination of ethylcellulose and mixture of hydroxypropylmethyl cellulose and polyethylene glycol.

In yet another embodiment the present invention provides an extended release matrix tablet comprising darifenacin or pharmaceutically acceptable salts thereof, incorporated in a hydrophobic matrix comprising one or more release controlling hydrophobic materials and one or more pharmaceutically acceptable excipients; wherein hydrophobic materials can be selected from group consisting of ethyl cellulose, cellulose acetate, cellulose acetate phthalate, polymethacrylates, calcium silicates, hydrogenated castor oil, hydrogenated vegetable oil, bees wax, carnauba wax and combination thereof; wherein drug release is solely controlled by hydrophobic matrix without dose dumping.

In yet another embodiment the present invention provides a process of preparing an extended release formulation comprising darifenacin or pharmaceutically acceptable salts thereof, one or more release controlling hydrophobic materials and one or more pharmaceutically acceptable excipients wherein process can be selected from direct compression, dry granulation, wet granulation (aqueous/non-aqueous or combination) and melt granulation.

The following examples illustrate preferred embodiments in accordance with the present invention without limiting the scope or spirit of the invention.

### Example No. 1

| | Formula | | |
|---|---|---|---|
| | A | B | C |
| | Strength | | |
| | 15 mg | | 7.5 mg |
| Ingredients | Quantity mg/Tablet | | |
| Darifenacin Hydrobromide eq. to | 17.858 | 17.858 | — |
| Darifenacin 15 mg | | | |
| Darifenacin Hydrobromide eq. to | — | — | 8.929 |
| Darifenacin 7.5 mg | | | |
| Lactose Monohydrate | 160.142 | 140.142 | 149.071 |
| Hydrogenated castor Oil | 20.000 | 40.000 | 40.000 |
| Magnesium Stearate | 2.000 | 2.000 | 2.000 |
| Total | 200.0 | 200.0 | 200.0 |

### Manufacturing Procedure

1. Sift Darifenacin Hydrobromide, Lactose monohydrate and Hydrogenated castor oil through suitable mesh.
2. Mix ingredients of step 1 for 10 Minutes.
3. Heat the dry mix of step 2 in at temperature NMT 90° C. under continuous stirring.
4. Cool the step 3 mixture under continuous stirring to form granules.
5. Pass the granules of step 4 through suitable mesh.
6. Lubricate step 5 granules with #40 passed magnesium stearate for 3 minutes in blender.
7. Compress the lubricated blend of step 6 into tablets by using suitable punches.

### Example No. 2

Formula D

| Strength | 15 mg |
|---|---|
| Ingredients | Quantity mg/Tablet |
| Darifenacin Hydrobromide eq. to | 17.858 |
| Darifenacin 15 mg | |
| Lactose Anhydrous | 133.142 |
| Hydrogenated Castor Oil | 45.000 |
| Talc | 2.000 |
| Magnesium Stearate | 2.000 |
| Total | 200.0 |

### Manufacturing Procedure

1. Sift Darifenacin Hydrobromide, Lactose Anhydrous and Hydrogenated castor oil through #40 mesh.
2. Mix Darifenacin Hydrobromide, part of lactose Anhydrous and Hydrogenated castor oil.
3. Sift blend of Step 2 through #40 mesh.
4. Mix blend of Step 3 and remaining quantity of Lactose Anhydrous for 10 minutes in blender.
5. Lubricate blend of step 4 with #40 passed Talc & Magnesium stearate for 3 minutes in blender.
6. Compress the lubricated blend of step 5 into tablets by using suitable punches.

### Example No. 3

Formula E

| Ingredients | Quantity mg/Tablet |
|---|---|
| Darifenacin Hydrobromide eq. to | 17.858 |
| Darifenacin 15 mg | |
| Lactose Anhydrous | 138.142 |
| Hydrogenated Vegetable Oil | 40.00 |
| Talc | 2.000 |
| Magnesium Stearate | 2.000 |
| Total | 200.0 |

### Manufacturing Procedure

1. Sift Darifenacin Hydrobromide, Lactose Anhydrous and Hydrogenated Vegetable Oil through #40 mesh.
2. Mix Darifenacin Hydrobromide and Lactose Anhydrous geometrically.
3. Sift blend of Step 2 through #40 mesh.
4. Mix blend of Step 3 and Hydrogenated Vegetable Oil for 10 minutes in blender.
5. Lubricate step 4 blend with #40 passed Talc & Magnesium stearate for 3 minutes in blender.
6. Compress the lubricated blend of step 5 into tablets by using suitable punches.

### Example No. 4

Formula F

| Ingredients | Quantity mg/Tablet |
|---|---|
| Darifenacin Hydrobromide eq. to | 17.858 |
| Darifenacin 15 mg | |
| Lactose Anhydrous | 123.142 |
| Hydrogenated Vegetable Oil | 40.00 |
| Ethyl Cellulose | 15.00 |
| Talc | 2.000 |
| Magnesium Stearate | 2.000 |
| Total | 200.0 |

### Manufacturing Procedure

1. Sift Darifenacin Hydrobromide, Lactose Anhydrous, Ethylcellulose and Hydrogenated Vegetable Oil through #40 mesh.
2. Mix Darifenacin Hydrobromide and Lactose Anhydrous geometrically.
3. Mix blend of Step 2 with Ethylcellulose and Hydrogenated Vegetable oil for 10 minutes in blender.
4. Add #40 mesh sieve passed Talc & Magnesium stearate to blend of Step 3 and mix for 3 minutes in blender.
5. Compress the lubricated blend of step 4 into tablets by using suitable punches.

### Example No. 5

Formula G

| Ingredients | Quantity mg/Tablet |
|---|---|
| Darifenacin Hydrobromide eq. to | 17.858 |
| Darifenacin 15 mg | |
| Dibasic Calcium Phosphate, Anhydrous | 47.142 |
| Polyethylene Glycol | 125.000 |
| Ethyl Cellulose | 6.00 |
| Isopropyl Alcohol | q.s. |
| Talc | 2.000 |
| Magnesium Stearate | 2.000 |
| Total | 200.0 |

### Manufacturing Procedure

1. Sift Darifenacin Hydrobromide, Dibasic Calcium Phosphate Anhydrous, Polyethylene Glycol and Ethylcellulose through #40 mesh sieve.
2. Mix Darifenacin Hydrobromide and Dibasic Calcium Phosphate Anhydrous geometrically.
3. Sift blend of step 2 through #40 mesh sieve.
4. Mix blend of Step 3 with Ethylcellulose and Polyethylene Glycol.
5. Granulate blend of step 4 by using sufficient quantity of Isopropyl Alcohol.
6. Dry the granules of step 5.
7. Pass the dried granules of Step 6 through #30 mesh sieve.
8. Add #40 mesh sieve passed Talc & Magnesium stearate to granules of Step 3 and mix.
9. Compress the lubricated blend of step 8 into tablets by using suitable punches.

### Example No 6

Formula H

| Ingredients | Quantity (mg/Tablet) |
|---|---|
| Darifenacin Hydrobromide eq. to | 17.858 |
| Darifenacin 15 mg | |
| Lactose Monohydrate | 140.142 |
| Ethylcellulose | 20.000 |
| Hydrogenated Vegetable Oil | 20.000 |
| Ethanol | q.s |
| Magnesium Stearate | 2.000 |
| Total | 200.0 |

### Manufacturing Procedure

Darifenacin hydrobromide, lactose monohydrate were mixed geometrically and sifted through suitable sieve and mixed with sifted ethylcellulose and hydrogenated vegetable oil and again sifted through suitable sieve and mixed. The blend was granulated with ethanol, dried and sifted through suitable sieve. The blend was lubricated with sifted magnesium stearate. The lubricated blend was compressed into tablets by using suitable punches.

### Example No. 7

Formula I

| Ingredients | Quantity (mg/Tablet) |
|---|---|
| Darifenacin Hydrobromide eq. to | 17.858 |
| Darifenacin 15 mg | |
| Dicalcium Phosphate, Anhydrous | 140.142 |
| Ethylcellulose | 20.000 |
| Hydrogenated Vegetable Oil | 20.000 |
| Ethanol | q.s |
| Magnesium Stearate | 2.000 |
| Total | 200.0 |

### Manufacturing Procedure

Darifenacin hydrobromide, dicalcium phosphate anhydrous were mixed geometrically and sifted through suitable sieve and mixed with sifted ethylcellulose and hydrogenated vegetable oil and again sifted through suitable sieve and mixed. The blend was granulated with ethanol, dried and sifted through suitable sieve. The blend was lubricated with sifted magnesium stearate. The lubricated blend was compressed into tablets by using suitable punches.

### Example No. 8

Formula J

| | Quantity (mg/Tablet) |
|---|---|
| Ingredients | |
| Darifenacin Hydrobromide eq. to | 17.858 |
| Darifenacin 15 mg | |
| Lactose Monohydrate | 138.142 |
| Hydrogenated Vegetable Oil | 42.000 |
| Magnesium Stearate | 2.000 |
| Total | 200.0 |
| Controlled Release Coating | |
| Ethylcellulose | 4.5 |
| Opadry ® Clear 03K19229 | 1.5 |
| Ethanol: IPA: Purified Water | q.s. |
| Total weight of coated tablet | 206.0 |

### Manufacturing Procedure

Darifenacin hydrobromide, lactose monohydrate and hydrogenated vegetable oil were mixed geometrically. The geometrically mixed blend was mixed in blender for 15 minutes and sifted through suitable sieve and again mixed in blender for 15 minutes. The blend was lubricated with sifted magnesium stearate. The lubricated blend was compressed into tablets by using suitable punches.

The tablets were coated with the solution of Ethylcellulose and Opadry® Clear prepared in ethanol:Isopropyl alcohol:purified water (40:40:20) to a desired weight gain.

### Example No. 9

Formula K

| Ingredients | Quantity (mg/Tablet) |
|---|---|
| Darifenacin Hydrobromide eq. to | 17.858 |
| Darifenacin 15 mg | |
| Dicalcium Phosphate, Anhydrous | 140.142 |
| Hydrogenated Vegetable Oil | 40.000 |
| Magnesium Stearate | 2.000 |
| Total | 200.0 |

### Manufacturing Procedure

Darifenacin hydrobromide, dicalcium phosphate anhydrous and hydrogenated vegetable oil was mixed geometrically. The geometrically mixed blend was mixed in blender for 10 minutes and sifted through suitable sieve and again mixed in blender for 10 minutes. The blend was lubricated with sifted magnesium stearate. The lubricated blend was compressed into tablets by using suitable punches.

### Example No. 10

Formula L

| Ingredients | Quantity (mg/Tablet) |
|---|---|
| Darifenacin Hydrobromide eq. to | 17.858 |
| Darifenacin 15 mg | |
| Lactose Monohydrate | 105.142 |
| Dicalcium Phosphate, Anhydrous | 35.000 |
| Hydrogenated Castor Oil | 40.000 |
| Magnesium Stearate | 2.000 |
| Total | 200.0 |

### Manufacturing Procedure

Darifenacin hydrobromide, lactose monohydrate, dicalcium phosphate anhydrous and hydrogenated castor oil were mixed geometrically, sifted through suitable sieve and mixed. The blend was again sifted through suitable sieve and mixed. The blend was lubricated with sifted magnesium stearate. The lubricated blend was compressed into tablets by using punches.

### Example No. 11

Formula M

| | Quantity (mg/Tablet) |
|---|---|
| Ingredients | |
| Darifenacin Hydrobromide eq. to | 17.858 |
| Darifenacin 15 mg | |
| Lactose Monohydrate | 140.142 |
| Hydrogenated Vegetable Oil | 40.000 |
| Magnesium Stearate | 2.000 |
| Total | 200.0 |
| Controlled Release Coating | |
| Ethylcellulose | 2.0 |
| PEG 6000 | 4.0 |
| Ethanol: IPA: Purified Water | q.s. |
| Total weight of coated tablet | 206.0 |

### Manufacturing Procedure

Darifenacin hydrobromide, lactose monohydrate and hydrogenated vegetable oil were mixed geometrically. The geometrically mixed blend was sifted through suitable sieve and mixed in blender for 15 minutes. The mixed blend was sifted through suitable sieve and again mixed in blender for 15 minutes. The blend was lubricated with sifted magnesium stearate in blender for 5 minutes. The lubricated blend was compressed into tablets by using suitable punches.

The tablets were coated with the solution of Ethylcellulose and PEG 6000 prepared in ethanol:Isopropyl alcohol:purified water (40:40:20) to a desired weight gain.

### Example No. 12

Formula N

| | Quantity (mg/Tablet) |
|---|---|
| Ingredients | |
| Darifenacin Hydrobromide eq. to | 17.858 |
| Darifenacin 15 mg | |
| Lactose Monohydrate | 140.142 |
| Hydrogenated Vegetable Oil | 40.000 |
| Magnesium Stearate | 2.000 |
| Total | 200.0 |
| Controlled Release Coating | |
| Ethylcellulose | 3.0 |
| Opadry ® Clear 03K19229 | 3.0 |
| Ethanol: IPA: Purified Water | q.s. |
| Total weight of coated tablet | 206.0 |

### Manufacturing Procedure

Darifenacin hydrobromide, lactose monohydrate and hydrogenated vegetable oil were mixed geometrically and sifted through suitable sieve and mixed in blender for 15 minutes. The blend was again sifted through suitable sieve and mixed in blender for 15 minutes. The blend was lubricated with sifted magnesium stearate in blender for 5 minutes. The lubricated blend was compressed into tablets by using suitable punches.

The tablets were coated with the solution of Ethylcellulose and Opadry® Clear prepared in ethanol:Isopropyl alcohol:purified water (40:40:20) to a desired weight gain.

### Example No. 13

Formula O

| Ingredients | Quantity (mg/Tablet) |
|---|---|
| Darifenacin Hydrobromide eq. to | 17.858 |
| Darifenacin 15 mg | |
| Lactose Monohydrate | 125.142 |
| Dicalcium Phosphate, Anhydrous (A-TAB) | 15.000 |
| Hydrogenated Castor Oil | 40.000 |
| Magnesium Stearate | 2.000 |
| Total | 200.0 |

### Manufacturing Procedure

Darifenacin hydrobromide, lactose monohydrate, dicalcium phosphate anhydrous and hydrogenated castor oil were mixed geometrically, sifted through suitable sieve and mixed. The blend was again sifted through suitable sieve and mixed. The blend was lubricated with sifted magnesium stearate. The lubricated blend was compressed into tablets by using suitable punches.

### Example No. 14

Formula P

| | Quantity (mg/Tablet) |
|---|---|
| Ingredients | |
| Darifenacin Hydrobromide eq. to | 17.858 |
| Darifenacin 15 mg | |
| Lactose Monohydrate | 120.142 |
| Tartaric Acid | 20.000 |
| Hydrogenated Vegetable Oil | 40.000 |
| Magnesium Stearate | 2.000 |
| Total | 200.0 |
| Controlled Release Coating | |
| Ethylcellulose | 4.0 |
| Opadry ® Clear 03K19229 | 4.0 |
| Ethanol: IPA: Purified Water | q.s. |
| Total weight of coated tablet | 208.0 |

### Manufacturing Procedure

Darifenacin hydrobromide, lactose monohydrate and hydrogenated vegetable oil were mixed geometrically and sifted through suitable sieve and mixed with sifted tartaric acid. The blend was again sifted through suitable sieve and mixed. The blend was lubricated with sifted magnesium stearate. The lubricated blend was compressed into tablets by using suitable punches.

The tablets were coated with the solution of Ethylcellulose and Opadry® Clear prepared in ethanol:Isopropyl alcohol:purified water (40:40:20) to a desired weight gain.

### Example No. 15

| | Formula | |
|---|---|---|
| | Q | R |
| | Quantity (mg/Tablet) | |
| Ingredients | | |
| Darifenacin Hydrobromide eq. to | 17.858 | — |
| Darifenacin 15 mg | | |
| Darifenacin Hydrobromide eq. to | — | 8.929 |
| Darifenacin 7.5 mg | | |
| Lactose Monohydrate | 140.142 | 149.071 |
| Hydrogenated Vegetable Oil | 40.000 | 40.0 |
| Magnesium Stearate | 2.000 | 2.0 |
| Total | 200.0 | 200.0 |
| Controlled Release Coating | | |
| Ethylcellulose | 4.0 | 4.0 |
| Opadry ® Clear YS-1R-7006 | 4.0 | 4.0 |
| Ethanol: IPA: Purified Water | q.s. | q.s. |
| Total weight of coated tablet | 208.0 | 208.0 |

### Manufacturing Procedure

Darifenacin hydrobromide, lactose monohydrate and hydrogenated vegetable oil were mixed geometrically. The geometrically mixed blend was sifted through suitable sieve and mixed in blender for 15 minutes. The mixed blend was again sifted through suitable sieve and again mixed in blender for 15 minutes. The blend was lubricated with sifted magnesium stearate in blender for 5 minutes and compressed into tablets by using suitable punches. The tablets were coated with the solution of Ethylcellulose and Opadry® Clear prepared in ethanol:Isopropyl alcohol:purified water (40:40:20) to a desired weight gain.

The formulations of Example No. 1 to Example No. 15 were subjected to in-vitro dissolution studies and the results obtained in comparison with Enablex 7.5 mg & 15 mg are presented below in Table No. 1, Table No. 2 and Table No. 3.

TABLE NO. 1
Comparative Dissolution profile in 0.01M HCl

| | Formula | | | | | | | | | | | | | | | |
|---|---|---|---|---|---|---|---|---|---|---|---|---|---|---|---|---|
| | Enablex | A | B | D | E | F | G | H | I | J | K | N | P | Q | Enablex | C |
| | Strength | | | | | | | | | | | | | | | |
| | 15 | 15 | 15 | 15 | 15 | 15 | 15 | 15 | 15 | 15 | 15 | 15 | 15 | 15 | 7.5 | 7.5 |
| | mg | mg | mg | mg | mg | mg | mg | mg | mg | mg | mg | mg | mg | mg | mg | mg |
| | Dissolution Condition | | | | | | | | | | | | | | | |
| Time | 900 ml, 0.01M HCl, USP Type I (Basket), 100 RPM | | | | | | | | | | | | | | | |
| (Hours) | Cumulative % Drug Released | | | | | | | | | | | | | | | |
| 1 | 11 | 35 | 22 | 19 | 18 | 23 | 44 | 23 | 7 | 1 | 14 | 16 | 20 | 17 | 13 | 22 |
| 2 | 18 | 47 | 33 | 28 | 27 | 33 | 65 | 32 | 10 | 6 | 19 | 25 | 31 | 27 | 24 | 33 |
| 4 | 32 | 70 | 47 | 38 | 40 | 46 | 88 | 44 | 15 | 15 | 27 | 36 | 43 | 44 | 41 | 48 |
| 6 | 46 | 81 | 59 | 47 | 50 | 53 | 100 | 53 | 19 | 27 | 34 | 48 | 54 | 57 | 56 | 61 |
| 8 | 60 | 95 | 60 | 59 | 58 | 63 | — | 60 | 22 | 39 | 39 | 57 | 65 | 73 | 68 | 65 |
| 10 | 72 | 102 | 76 | 64 | 65 | 69 | — | 66 | 24 | 46 | 43 | 64 | 71 | 77 | 76 | 78 |
| 12 | 81 | — | 83 | 72 | 71 | 76 | — | 71 | 27 | 54 | 48 | 73 | 83 | 84 | 87 | 85 |
| 16 | 95 | — | 95 | 82 | 83 | 84 | — | 79 | 32 | 64 | 55 | 86 | 93 | 92 | 99 | 95 |
| 20 | 102 | — | 104 | 85 | 91 | 91 | — | 84 | 35 | 74 | 60 | 94 | 101 | 96 | 106 | 103 |
| 24 | 106 | — | 104 | — | 99 | 97 | — | 89 | 40 | 81 | 65 | 101 | 102 | 100 | 110 | 107 |

TABLE NO. 2
Comparative Dissolution profile in pH 6.8 phosphate buffer

| | Formula | | | | | | | |
|---|---|---|---|---|---|---|---|---|
| | Enablex | L | M | N | P | Q | Enablex | R |
| | Strength | | | | | | | |
| | 15 | 15 | 15 | 15 | 15 | 15 | 7.5 | 7.5 |
| | mg | mg | mg | mg | mg | mg | mg | mg |
| | Dissolution Condition | | | | | | | |
| Time | 900 ml, pH 6.8 Phosphate Buffer, USP Type I (Basket), 100 RPM | | | | | | | |
| (Hours) | Cumulative % Drug Released | | | | | | | |
| 1 | 9 | 10 | 11 | 8 | 19 | 10 | 9 | 12 |
| 2 | 15 | 14 | 17 | 13 | 31 | 19 | 19 | 21 |
| 4 | 27 | 22 | 26 | 20 | 45 | 30 | 32 | 34 |
| 6 | 37 | 30 | 35 | 25 | 54 | 40 | 45 | 49 |
| 8 | 46 | 34 | 44 | 29 | 59 | 48 | 55 | 59 |
| 10 | 53 | 38 | 49 | 34 | 64 | 53 | 63 | 64 |
| 12 | 60 | 43 | 56 | 37 | 68 | 59 | 72 | 67 |
| 16 | 70 | 47 | 63 | 45 | 73 | 67 | 83 | 73 |
| 20 | 78 | 55 | 69 | 55 | 75 | 71 | 93 | 77 |
| 24 | 84 | — | 74 | 63 | 78 | 78 | 101 | 79 |

TABLE NO. 3
Comparative Multimedia dissolution profile of Q with Enablex.

| | Formula | | | | | | | |
|---|---|---|---|---|---|---|---|---|
| | Enablex | Q | Enablex | Q | Enablex | Q | Enablex | Q |
| | Media | | | | | | | |
| | | | pH 4.5 | | pH 6.8 | | | |
| | 0.01M HCl | | Acetate buffer | | Phosphate buffer | | Water | |
| | Condition | | | | | | | |
| Time | 900 ml, USP Type I (Basket), 100 RPM | | | | | | | |
| (hours) | Cumulative % Drug Released | | | | | | | |
| 1 | 13 | 17 | 13 | 25 | 9 | 11 | 12 | 13 |
| 2 | 23 | 27 | 22 | 29 | 15 | 19 | 21 | 21 |
| 4 | 40 | 44 | 38 | 44 | 27 | 30 | 35 | 33 |
| 6 | 54 | 57 | 50 | 57 | 37 | 40 | 47 | 44 |
| 8 | 67 | 73 | 61 | 67 | 46 | 48 | 57 | 53 |
| 10 | 77 | 77 | 70 | 77 | 53 | 53 | 66 | 62 |
| 12 | 86 | 84 | 77 | 84 | 60 | 59 | 74 | 69 |
| 16 | 98 | 92 | 89 | 95 | 70 | 67 | 84 | 77 |
| 20 | 104 | 96 | 95 | 100 | 78 | 71 | 91 | 81 |
| 24 | 105 | 100 | 99 | 101 | 84 | 78 | 94 | 83 |

We claim:

1. An extended release formulation comprising;
darifenacin or pharmaceutically acceptable salts thereof and one or more release controlling hydrophobic materials.

2. An extended release formulation according to claim 1 wherein darifenacin or pharmaceutically acceptable salts thereof is present from about 1.5% w/w to about 50% w/w of formulation.

3. An extended release formulation according to claim 1 wherein hydrophobic material can be selected from group consisting of ethyl cellulose, cellulose acetate, cellulose acetate phthalate, polymethacrylates, calcium silicates, hydrogenated castor oil, hydrogenated vegetable oil, bees wax, carnauba wax and combination thereof.

4. An extended release formulation according to claim 1 wherein hydrophobic material is present from about 10% w/w to about 98% w/w of formulation.

5. An extended release formulation according to claim 1 wherein formulation is prepared using direct compression, dry granulation, wet granulation (aqueous/non-aqueous or combination) or melt granulation.

6. An extended release formulation according to claim 1 wherein formulation is optionally coated with one or more release controlling materials.

7. An extended release formulation according to claim 1 wherein an in-vitro dissolution rate when measured using the USP Type I (Basket Apparatus) at 100 rpm in 900 ml, 0.01M hydrochloric acid at 37° C.;
less than 40% darifenacin released after 1 hour;
from about 25% to about 60% darifenacin released after 4 hours;
from about 50% to about 85% darifenacin released after 8 hours;
from about 55% to about 100% darifenacin released after 12 hours;
from about 70% to about 100% darifenacin released after 16 hours;
and greater than 85% darifenacin released after 24 hours.

8. An extended release formulation comprising darifenacin or pharmaceutically acceptable salts thereof, wherein darifenacin is incorporated in a matrix comprising one or more release controlling hydrophobic materials and one or more release controlling hydrophilic materials.

9. An extended release formulation according to claim 8 wherein darifenacin or pharmaceutically acceptable salts thereof is present from about 1.5% w/w to about 50% w/w of formulation.

10. An extended release formulation according to claim 8 wherein hydrophobic materials can be selected from group consisting of ethyl cellulose, cellulose acetate, cellulose acetate phthalate, polymethacrylates, calcium silicates, hydrogenated castor oil, hydrogenated vegetable oil, bees wax, carnauba wax and combination thereof.

11. An extended release formulation according to claim 8 wherein hydrophilic materials can be selected from group consisting of hydroxypropylmethyl cellulose, hydroxypropyl cellulose, methylcellulose, povidone, polyethylene glycols, vinyl acetate copolymers, polysaccharides such as alginates, xanthan gum, chitosan, carrageenan, dextran, polyalkylene oxides such as polyethylene oxide, methacrylic acid copolymers, maleic anhydride/methyl vinyl ether copolymers, carbomers and combinations thereof.

12. An extended release formulation according to claim 8 wherein hydrophobic material is present from about 10% w/w to about 98% w/w of formulation.

13. An extended release formulation according to claim 8 wherein formulation is prepared using direct compression, dry granulation, wet granulation (aqueous/non-aqueous or combination) or melt granulation.

14. An extended release formulation according to claim 8 wherein formulation is optionally coated with one or more release controlling materials.

15. An extended release formulation according to claim 8 wherein an in-vitro dissolution rate when measured using the USP Type I (Basket Apparatus) at 100 rpm in 900 ml, 0.01M hydrochloric acid at 37° C.;
less than 40% darifenacin released after 1 hour;
from about 25% to about 60% darifenacin released after 4 hours;
from about 50% to about 85% darifenacin released after 8 hours;
from about 55% to about 100% darifenacin released after 12 hours;
from about 70% to about 100% darifenacin released after 16 hours;
and greater than 85% darifenacin released after 24 hours.

16. An extended release formulation comprising darifenacin or pharmaceutically acceptable salts thereof, wherein darifenacin is incorporated in a matrix comprising one or more release controlling hydrophobic materials optionally coated with combination of one or more release controlling hydrophobic materials and one or more release controlling hydrophilic materials.

17. An extended release formulation according to claim 16 wherein darifenacin or pharmaceutically acceptable salts thereof is present from about 1.5% w/w to about 50% w/w of formulation.

18. An extended release formulation according to claim 16 wherein hydrophobic materials can be selected from group consisting of ethyl cellulose, cellulose acetate, cellulose acetate phthalate, polymethacrylates, calcium silicates, hydrogenated castor oil, hydrogenated vegetable oil, bees wax, carnauba wax and combination thereof.

19. An extended release formulation according to claim 16 wherein hydrophilic material can be selected from group consisting of hydroxypropylmethyl cellulose, hydroxypropyl cellulose, methylcellulose, povidone, polyethylene glycols, vinyl acetate copolymers, polysaccharides such as alginates, xanthan gum, chitosan, carrageenan, dextran, polyalkylene oxides such as polyethylene oxide, methacrylic acid copolymers, maleic anhydride/methyl vinyl ether copolymers, carbomers and combinations thereof.

20. An extended release formulation according to claim 16 wherein hydrophobic material is present from about 10% w/w to about 98% w/w of formulation.

21. An extended release formulation according to claim 16 wherein formulation is prepared using direct compression, dry granulation, wet granulation (aqueous/non-aqueous or combination) or melt granulation.

22. An extended release formulation according to claim 16 wherein an in-vitro dissolution rate when measured using the USP Type I (Basket Apparatus) at 100 rpm in 900 ml, 0.01M hydrochloric acid at 37° C.;
less than 40% darifenacin released after 1 hour;
from about 25% to about 60% darifenacin released after 4 hours;
from about 50% to about 85% darifenacin released after 8 hours;
from about 55% to about 100% darifenacin released after 12 hours;
from about 70% to about 100% darifenacin released after 16 hours;
and greater than 85% darifenacin released after 24 hours

23. An extended release matrix tablet comprising darifenacin or pharmaceutically acceptable salts thereof, wherein darifenacin is incorporated in a hydrophobic matrix comprising one or more release controlling hydrophobic materials and one or more pharmaceutically acceptable excipients.

24. An extended release matrix tablet according to claim 23 wherein darifenacin or pharmaceutically acceptable salts thereof is present from about 1.5% w/w to about 50% w/w of formulation.

25. An extended release matrix tablet according to claim 23 wherein hydrophobic materials can be selected from group consisting of ethyl cellulose, cellulose acetate, cellulose acetate phthalate, polymethacrylates, calcium silicates, hydrogenated castor oil, hydrogenated vegetable oil, bees wax, carnauba wax and combination thereof.

26. An extended release matrix tablet according to claim 23 wherein hydrophobic material is present from about 10% w/w to about 98% w/w of formulation.

27. An extended release matrix tablet according to claim 23 wherein formulation is prepared by using direct compression, dry granulation, wet granulation (aqueous/non-aqueous or combination) or melt granulation.

28. An extended release formulation according to claim 23 wherein formulation is optionally coated with one or more release controlling materials.

29. An extended release formulation according to claim 23 wherein an in-vitro dissolution rate when measured using the USP Type I (Basket Apparatus) at 100 rpm in 900 ml, 0.01M hydrochloric acid at 37° C.;
less than 40% darifenacin released after 1 hour;
from about 25% to about 60% darifenacin released after 4 hours;
from about 50% to about 85% darifenacin released after 8 hours;
from about 55% to about 100% darifenacin released after 12 hours;
from about 70% to about 100% darifenacin released after 16 hours;
and greater than 85% darifenacin released after 24 hours.

30. An extended release matrix tablet comprising darifenacin or pharmaceutically acceptable salts thereof comprising;
a) from about 1.5% w/w to about 50% w/w of formulation darifenacin or pharmaceutically acceptable salts thereof;
b) from about 10% w/w to about 98% w/w of formulation one or more hydrophobic materials;
c) one or more pharmaceutically acceptable excipients;
d) optionally coated with combination of one or more hydrophobic materials and one or more hydrophilic materials.

31. An extended release matrix tablet according to claim 30 wherein an in-vitro dissolution rate when measured using the USP Type I (Basket Apparatus) at 100 rpm in 900 ml, 0.01M hydrochloric acid at 37° C. less than 40% darifenacin released after 1 hour;
from about 25% to about 65% darifenacin released after 4 hours;
from about 50% to about 85% darifenacin released after 8 hours;
from about 55% to about 100% darifenacin released after 12 hours;
from about 70% to about 100% darifenacin released after 16 hours;
and greater than 85% darifenacin released after 24 hours.

32. An extended release matrix tablet comprising;
a) from about 1.5% to about 50% w/w of formulation darifenacin or pharmaceutically acceptable salts thereof;
b) from about 10% w/w to about 98% w/w of formulation of hydrogenated vegetable oil or hydrogenated castor oil;
c) one or more pharmaceutically acceptable excipients;
d) optionally coated with combination of ethylcellulose and mixture of hydroxypropylmethyl cellulose and polyethylene glycol.

33. An extended release matrix tablet according to claim 32 wherein an in-vitro dissolution rate when measured using the USP Type 1 (Basket Apparatus) at 100 rpm in 900 ml, 0.01M hydrochloric acid at 37° C. less than 40% darifenacin released after 1 hour;
from about 25% to about 65% darifenacin released after 4 hours;
from about 50% to about 85% darifenacin released after 8 hours;
from about 55% to about 100% darifenacin released after 12 hours;
from about 70% to about 100% darifenacin released after 16 hours;
and greater than 85% darifenacin released after 24 hours.
